(19) United States
(12) Patent Application Publication
(10) Pub. No.: US 2016/0051134 A1
(43) Pub. Date: Feb. 25, 2016

(54) GUIDANCE OF THREE-DIMENSIONAL SCANNING DEVICE

(71) Applicant: United Sciences, LLC, Atlanta, GA (US)

(72) Inventor: Karol Hatzilias, Atlanta, GA (US)

(21) Appl. No.: 14/462,619

(22) Filed: Aug. 19, 2014

(51) Int. Cl. A61B 1/227

(52) U.S. Cl. CPC A61B 1/227; H04N 2005/2255

(57) ABSTRACT

Disclosed are various embodiments for providing operability and guidance of a mobile scanning device configured to scan and generate images and reconstructions of surfaces of objects. A mobile scanning device, such as an otoscanner, may be guided such that collection of data for the surfaces of objects is optimized. In addition, the mobile scanning device may be further guided such that a position of the mobile scanning device may be maintained utilizing detection of fiducial markers via one or more sensors.

### BACKGROUND

There are various needs for understanding the shape and size of cavity surfaces, such as body cavities. For example, hearing aids, hearing protection, custom head phones, and wearable computing devices can use impressions of a patient's ear canal or similar body cavities. To construct an impression of an ear canal, audiologists have injected a silicone material into a patient's ear canal, waited for the material to harden, and then provided the mold to manufacturers who use the resulting silicone impression to create a custom fitting in-ear device. As may be appreciated, the process is slow, expensive, and unpleasant for the patient as well as a medical professional performing the procedure.

Computer vision and photogrammetry generally relates to acquiring and analyzing images in order to produce data by electronically understanding an image using various algorithmic methods. For example, computer vision may be employed in event detection, object recognition, motion estimation, and various other tasks. Object detection and collision recognition in devices utilizing computer vision remains problematic.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1A-1C are drawings of an otoscanner according to various embodiments of the present disclosure.

FIG. 2 is a pictorial diagram of an example user interface rendered on a display in data communication with the otoscanner of FIGS. 1A-1C according to various embodiments of the present disclosure.

FIG. 3 is a drawing of a fiducial marker that may be used by the otoscanner of FIGS. 1A-1C in pose estimation and position determination according to various embodiments of the present disclosure.

FIG. 4 is a drawing of the otoscanner of FIGS. 1A-1C conducting a scan of an ear encompassed by the fiducial marker of FIG. 3 that may be used in pose estimation according to various embodiments of the present disclosure.

FIG. 5 is a drawing of a camera model that may be employed in an estimation of a pose of the scanning device of FIGS. 1A-1C according to various embodiments of the present disclosure.

FIG. 6 is a drawing of a partial bottom view of the otoscanner of FIGS. 1A-1C according to various embodiments of the present disclosure.

FIG. 7 is a drawing illustrating the epipolar geometric relationships of at least two imaging devices in data communication with the otoscanner of FIGS. 1A-1C according to various embodiments of the present disclosure.

FIGS. 8A-B are pictorial diagrams of example user interfaces rendered on a display in data communication with the otoscanner of FIGS. 1A-1C according to various embodiments of the present disclosure.

FIGS. 9A-B are pictorial diagrams of examples of a user interface rendered on a display in data communication with the otoscanner of FIGS. 1A-1C according to various embodiments of the present disclosure.

FIG. 10 is a flowchart illustrating one example of functionality implemented as portions of a guidance system application executed in the otoscanner of FIGS. 1A-1C according to various embodiments of the present disclosure.

FIG. 11 is another flowchart illustrating one example of functionality implemented as portions of a guidance system application executed in the otoscanner of FIGS. 1A-1C according to various embodiments of the present disclosure.

FIG. 12 is a schematic block diagram that provides one example illustration of a computing environment employed in the otoscanner of FIGS. 1A-1C according to various embodiments of the present disclosure.

### DETAILED DESCRIPTION

The present disclosure relates to operability and guidance of a mobile scanning device configured to scan and generate images and reconstructions of surfaces. Advancements in computer vision permit imaging devices, such as digital cameras, to be employed as sensors useful in determining locations, shapes, and appearances of objects in a three-dimensional space. For example, a position and an orientation of an object in a three-dimensional space may be determined utilizing digital images obtained via various image capturing devices. As may be appreciated, the position and orientation of the object in the three-dimensional space may be beneficial in generating additional data about the object, or about other objects, in the same three-dimensional space.

For example, mobile scanning devices capable of handheld operation may be used in various industries to scan objects to generate data pertaining to the objects being scanned. A mobile scanning device can employ an imaging device, such as a camera, to determine information about the object being scanned, such as the size, shape, or structure of the object, the distance of the object from the scanning device, etc. As a non-limiting example, a mobile scanning device may include an otoscanner configured to visually inspect or scan the ear canal of a human or animal. An otoscanner may comprise one or more cameras that may be beneficial in generating data about the ear canal subject of the scan, such as the size, shape, or structure of the ear canal. This data may be used in generating three-dimensional reconstructions of the ear canal that may be useful in customizing in-ear devices, for example but not limited to, hearing aids, in-the-ear headphones, or wearable computing devices.

Data about the surfaces or surface cavities subject to the scan can be obtained via an otoscanner or similar scanning device using sensors, such as imaging devices, fan lights, etc., to record precise measurements of the object being subjected to the scan. Multiple scans or “sweeps” of the surface using the scanning device may be needed to obtain a complete set of data providing accurate information about the surface cavity being subjected to the scan. Such data can be used in generating an accurate three-dimensional reconstruction of, e.g., the ear canal. Obtaining a complete set of data while minimizing the number of sweeps remains problematic.

Accordingly, a guidance system may be employed in a scanning device, such as an otoscanner, to facilitate an initial scan of a surface also referred to as a “ghost scan.” Data obtained during the ghost scan can be employed by the guidance system to provide a user of the scanning device with directions as how to optimally operate the scanning device such that the data obtained during a sweep is optimized, thereby reducing a need for subsequent sweeps to obtain missing or incomplete data. In addition, the guidance system may facilitate maintaining a field of fiducial vision between the scanning device and at least one fiducial marker employed to facilitate tracking of the scanning device in a three-dimensional space. In the following discussion, a general description of the guidance system and its components is provided, followed by a discussion of the operation of the same.

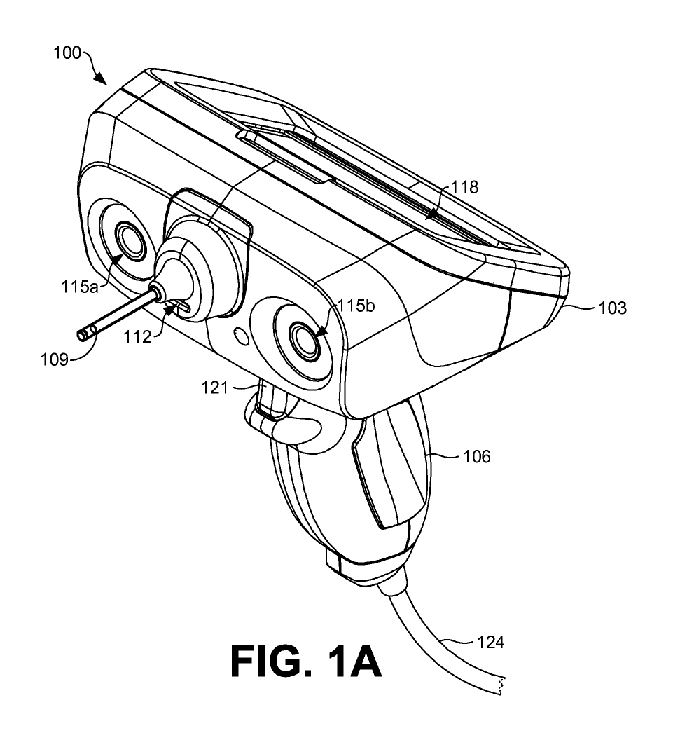

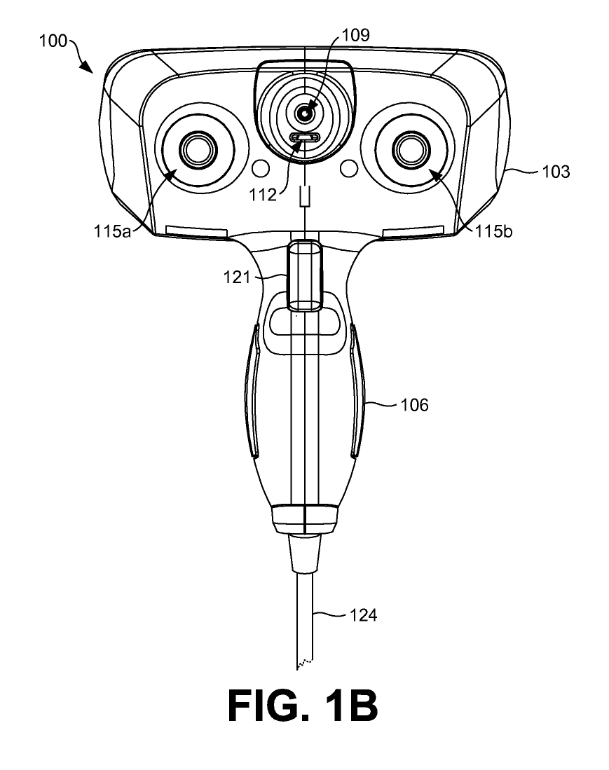

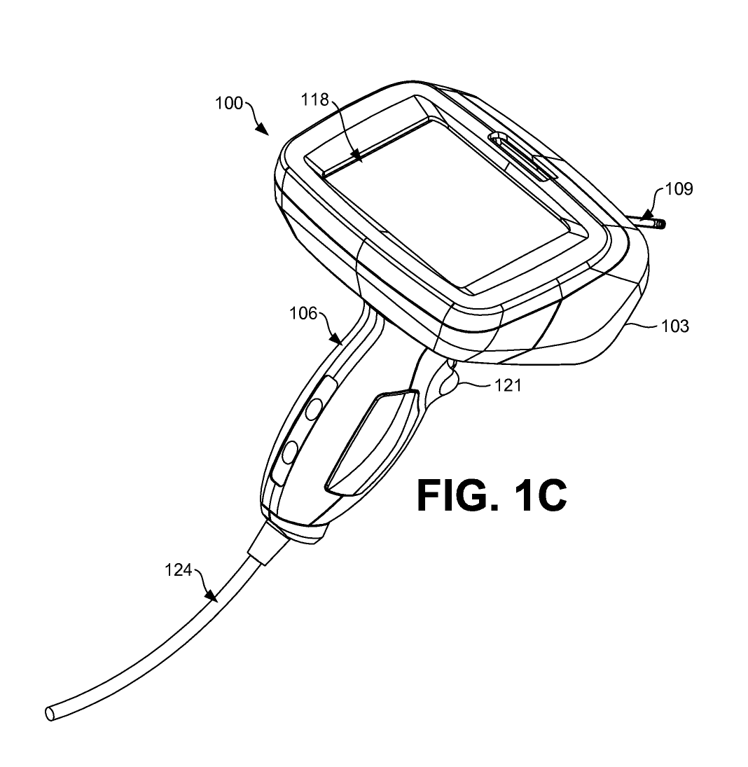

With reference to FIG. 1A, shown is an example drawing of a scanning device 100 according to various embodiments of the present disclosure. The scanning device 100, as illustrated in FIG. 1A, may comprise, for example, a body 103 and a hand grip 106. Mounted upon the body 103 of the scanning device 100 are a probe 109, a fan light element 112, and a plurality of tracking sensors comprising, for example, a first imaging device 115a and a second imaging device 115b. According to various embodiments, the scanning device 100 may further comprise a display screen 118 configured to render a user interface comprising, for example, a feed of images captured via the probe 109, the first imaging device 115a, the second imaging device 115b, and/or other imaging devices.

The hand grip 106 may be configured such that the length is long enough to accommodate large hands and the diameter is small enough to provide enough comfort for smaller hands. A trigger 121, located within the hand grip 106, may perform various functions such as initiating a scan of a surface, controlling a user interface rendered on the display, and/or otherwise modifying the function of the scanning device 100.

The scanning device 100 may further comprise a cord 124 that may be employed to communicate data signals to external computing devices and/or to power the scanning device 100. As may be appreciated, the cord 124 may be detachably attached to facilitate the mobility of the scanning device 100 when held in a hand via the hand grip 106. According to various embodiments of the present disclosure, the scanning device 100 may not comprise a cord 124, thus acting as a wireless and mobile device capable of wireless communication via, for example, Bluetooth, ZigBee, Induction Wireless, Infrared Wireless, Ultra Wideband, Wireless Fidelity (Wi-Fi), or any other similar communication medium.

The probe 109 mounted on the scanning device 100 may be configured to guide light received at a proximal end of the probe 109 to a distal end of the probe 109 and may be employed in the scanning of a surface cavity, such as an ear canal, by placing the probe 109 near or within the surface cavity. During a scan, the probe 109 may be configured to project a 360-degree ring onto the cavity surface and capture reflections from the projected ring to capture data that may be used to reconstruct the size and shape of the surface cavity. In addition, the scanning device 100 may be configured to capture video images of the cavity surface by projecting video illuminating light onto the cavity surface and capturing video images of the cavity surface.

The fan light element 112 mounted onto the scanning device 100 may be configured to emit light in a fan line for scanning an outer surface. The fan light element 112 comprises a fan light source projecting light onto a single element lens to collimate the light and generate a fan line for scanning the outer surface. By using triangulation of the reflections captured when projected onto a surface, the imaging sensor within the scanning device 100 can reconstruct the scanned surface.

FIG. 1A illustrates an example of a first imaging device 115a and a second imaging device 115b mounted on or within the body 103 of the scanning device 100, for example, in an orientation that is opposite from the display screen 118. The display screen 118, as will be discussed in further detail below, may be configured to render digital media of a surface cavity captured by the scanning device 100 in a user interface as the probe 109 is moved within the cavity. The display screen 118 can also display, either separately or simultaneously, real-time constructions of three-dimensional images corresponding to the scanned cavity.

Referring next to FIG. 1B, shown is another drawing of the scanning device 100 according to various embodiments. In this example, the scanning device 100 comprises a body 103, a probe 109, a hand grip 106, a fan light element 112, a trigger 121, and a cord 124 (optional), all implemented in a fashion similar to that of the scanning device described above with reference to FIG. 1A. In the examples of FIGS. 1A and 1B, the scanning device 100 is implemented with the first imaging device 115a and the second imaging device 115b mounted within the body 103 without hindering or impeding a view of the first imaging device 115a and/or a second imaging device 115b. According to various embodiments of the present disclosure, the placement of the imaging devices 115 may vary as needed to facilitate accurate pose estimation, as will be discussed in greater detail below.

Turning now to FIG. 1C, shown is another drawing of the scanning device 100 according to various embodiments. In the non-limiting example of FIG. 1C, the scanning device 100 comprises a body 103, a probe 109, a hand grip 106, a trigger 121, and a cord 124 (optional), all implemented in a fashion similar to that of the scanning device described above with reference to FIGS. 1A-1B.

In the examples of FIGS. 1A, 1B, and 1C, the scanning device 100 is implemented with the probe 109 mounted on the body 103 between the hand grip 106 and the display screen 118. The display screen 118 is mounted on the opposite side of the body 103 from the probe 109 and distally from the hand grip 106. To this end, when an operator takes the hand grip 106 in the operator's hand and positions the probe 109 to scan a surface, both the probe 109 and the display screen 118 are easily visible to the operator.

Further, the display screen 118 is coupled for data communication with the imaging devices 115 (not shown). The display screen 118 may be configured to display and/or render images of the scanned surface. The displayed images may include digital images or video of the cavity captured via the probe 109 and the fan light element 112 (not shown) as the probe 109 is moved within the cavity. The images shown on the display, for example, via a user interface, may also include real-time reconstructions of three-dimensional images corresponding to the scanned cavity. The display screen 118 may be configured to display, either separately or simultaneously, the video images and the three-dimensional images.

According to various embodiments of the present disclosure, the imaging devices 115 of FIGS. 1A, 1B, and 1C, may comprise a variety of cameras to capture one or more digital images of a surface cavity subject to a scan. A camera is described herein as a ray-based sensing device and may comprise, for example, a charge-coupled device (CCD) camera, a complementary metal-oxide semiconductor (CMOS) camera, or any other appropriate camera. Similarly, the camera employed as an imaging device 115 may comprise one of a variety of lenses such as: apochromat (APO), process with pincushion distortion, process with barrel distortion, fisheye, stereoscopic, soft-focus, infrared, ultraviolet, swivel, shift, wide angle, any combination thereof, and/or any other appropriate type of lens.

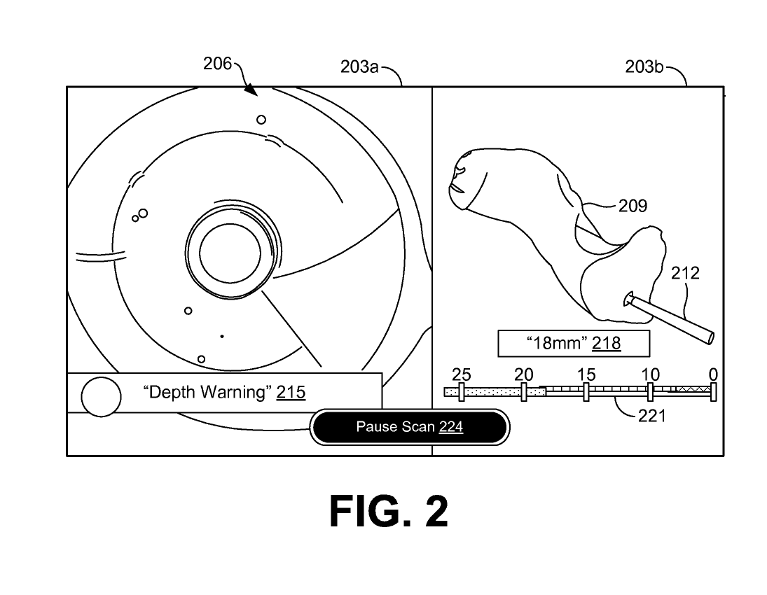

Referring next to FIG. 2, shown is an example of a user interface that may be rendered, for example, on a display screen 118 within the scanning device 100 or in any other display in data communication with the scanning device 100. In the non-limiting example of FIG. 2, a user interface may comprise a first portion 203a and a second portion 203b rendered separately or concurrently in a display. For example, in the first portion 203a, a real-time video stream may be rendered, providing an operator of the scanning device 100 with a view of a surface cavity being scanned. The real-time video stream may be generated via the probe 109 or via one of the imaging devices 115.

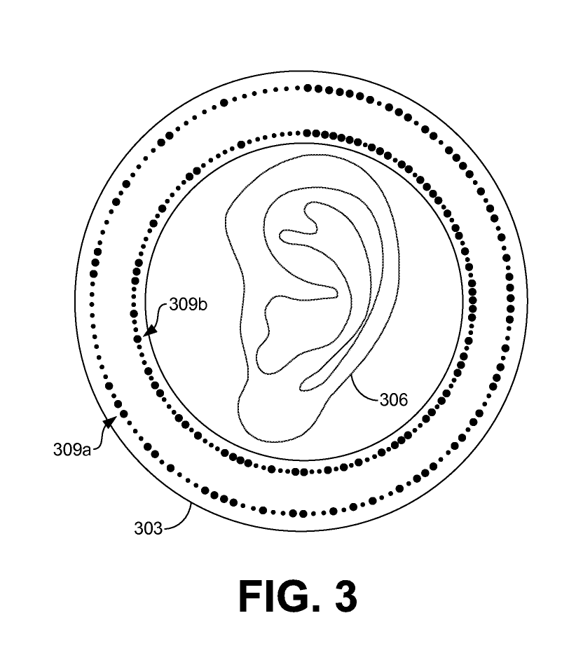

In the second portion 203b, a real-time three-dimensional reconstruction of the object being scanned may be rendered, providing the operator of the scanning device 100 with an estimate regarding what portion of the surface cavity has been scanned. For example, the three-dimensional reconstruction may be non-existent as a scan of a surface cavity is initiated by the operator. As the operator progresses in conducting one or more sweeps of the surface and/or surface cavity, a three-dimensional reconstruction of the surface cavity may be generated portion-by-portion, progressing into a complete reconstruction of the surface and/or surface cavity at the completion of the scan. In the non-limiting example of FIG. 3, the first portion 203a may comprise, for example, an inner view of an ear canal 206 obtained via the probe 109 and the second portion 203b may comprise, for example, a three-dimensional reconstruction of an ear canal 209, or vice versa.

A three-dimensional reconstruction of an ear canal 209 may be generated via one or more processors internal to the scanning device 100, external to the scanning device 100, or a combination thereof. Generating the three-dimensional reconstruction of the object being subjected to the scan may require information related to the pose of the scanning device 100. The three-dimensional reconstruction of the ear canal 209 may further comprise, for example, a probe model 212 emulating a position of the probe 109 relative to the surface cavity being scanned by the scanning device. Determining the information that may be used in the three-dimensional reconstruction of the object being subjected to the scan and the probe model 212 will be discussed in greater detail below.

A notification area 215 may provide the operator of the scanning device with notifications, which can assist the operator with conducting a scan or warning the operator of potential harm to the object being scanned. The notification area 215 may further comprise, for example, notifications provided to the operator that provide feedback or instruction on how to optimize data collection. Measurements 218 may be rendered on the display to assist the operator in conducting scans of surface cavities at certain distances and/or depths. A bar 221 may provide the operator with an indication of which depths have been thoroughly scanned as opposed to which depths or distances remain to be scanned via placement, coloring, or highlighting of the respective portions of the bar 221 which may be recognized by the operator. One or more buttons 224 may be rendered at various locations on the user interface permitting the operator to initiate a scan of an object and/or manipulate the user interface presented on the display screen 118 or other display in data communication with the scanning device 100. According to one embodiment, the display screen 118 comprises a touch-screen display and the operator may engage the button 224 to pause and/or resume an ongoing scan.

Although portion 203a and portion 203b are shown simultaneously in a side-by-side arrangement, other embodiments may be employed without deviating from the scope of the user interface. For example, portion 203a may be rendered on the display screen 118 on the scanning device 100 and portion 203b may be located on a display external to the scanning device 100, and vice versa.

Turning now to FIG. 3, shown is an example drawing of a fiducial marker 303 that may be employed in pose estimation computed during a scan of an ear 306 or other surface. In the non-limiting example of FIG. 3, a fiducial marker 303 may comprise a first circle-of-dots 309a and a second circle-of-dots 309b (collectively or independently circle-of-dots 309) that form two rings circumnavigating the fiducial marker 303. Although shown as a circular arrangement, the fiducial marker 303 is not so limited, and may comprise alternatively an oval, square, elliptical, rectangular, or appropriate geometric arrangement. Moreover, although shown with two rings (first circle-of-dots 309a and second circle-of-dots 309b), a fiducial marker 303 may comprise one or more rings comprising a circle-of-dots pattern.

According to various embodiments of the present disclosure, a circle-of-dots 309 may comprise, for example, a combination of uniformly or variably distributed large dots and a small dots that, when detected, represent a binary number. For example, in the event seven dots in a circle-of-dots 309 are detected in a digital image, the sequence of seven dots may be analyzed to identify (a) the size of the dots and (b) a number or other identifier corresponding to the arrangement of the dots. Detection of a plurality of dots in a digital image may be employed using known region- or blob-detection techniques, as may be appreciated.

As a non-limiting example, a sequence of seven dots comprising small-small-large-small-large-large-large may represent an identifier represented as a binary number of 0-0-1-0-1-1-1 (or, alternatively, 1-1-0-1-0-0-0). The detection of this arrangement of seven dots, represented by the corresponding binary number, may be indicative of a pose of the scanning device 100 relative to the fiducial marker 303. For example, a lookup table may be employed to map the binary number to a pose estimate, providing at least an initial estimated pose that may be refined and/or supplemented using information inferred via one or more camera models, as will be discussed in greater detail below. Although the example described above employs a binary operation using a combination of small dots and large dots to form a circle-of-dots 309, variable size dots (having, for example, 11 sizes) may be employed using variable base numeral systems (for example, a base-fl numeral system).

The arrangement of dots in the second circle-of-dots 309b may be the same as the first circle-of-dots 309a, or may vary. If the second circle-of-dots 309b comprises the same arrangement of dots as the first circle-of-dots 309a, then the second circle-of-dots 309b may be used independently or collectively (with the first circle-of-dots 309a) to determine an identifier indicative of the pose of the scanning device 100. Similarly, the second circle-of-dots 309b may be used to determine an error of the pose estimate determined via the first circle-of-dots 309a, or vice versa.

Accordingly, a fiducial marker 303 may be placed relative to the object being scanned to facilitate in accurate pose estimation of the scanning device 100. In the non-limiting example of FIG. 3, the fiducial marker 303 may circumscribe or otherwise surround an ear 306, or other surface, subject to a scan via the scanning device 100. In one embodiment, the fiducial marker 303 may be detachably attached around the ear of a patient using a headband or similar means.

In other embodiments, a fiducial marker may not be needed, as the tracking targets may be naturally occurring features surrounding and/or within the cavity to be scanned, which are detectable by employing various computer vision techniques. For example, assuming that a person's ear is being scanned by the scanning device 100, the tracking targets may include, hair, folds of the ear, skin tone changes, freckles, moles, and/or any other naturally occurring feature on the person's head relative to the ear.

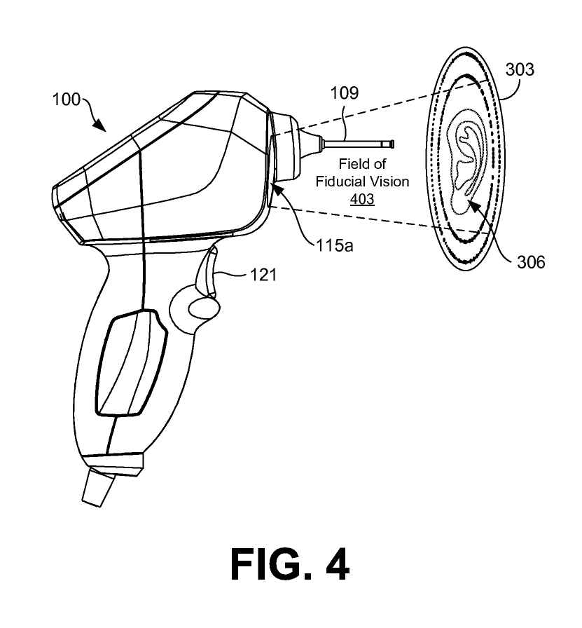

Moving on to FIG. 4, shown is an example of the scanning device 100 conducting a scan of an object, such as an ear 306. However, it should be noted that the scanning device 100 may be configured to scan other types of surfaces or cavities and is not limited to human or animal applications. During a scan, a first imaging device 115a and a second imaging device 115b (not shown) can capture digital images of the object being subjected to the scan. As described above with respect to FIG. 3, a fiducial marker 303 may circumscribe or otherwise surround the object being subjected to the scan. Thus, while an object is being scanned by the probe 109, the imaging devices 115 may capture images of the fiducial marker 303 that may be used in the determination of a pose of the scanning device 100, as discussed above with respect to FIG. 3. As the imaging devices 115 capture images of the fiducial marker 303, the probe 109 can capture data corresponding to the surface of the ear 306 as described above.

As may be appreciated, to accurately determine the pose of the scanning device 100, the imaging devices 115 must maintain a field of fiducial vision 403 with the fiducial marker 303. As the scanning device 100 is mobile and able to be held by a hand of the operator during a scan, the scanning device 100 may be prone to losing the field of fiducial vision 403, thus losing the ability to accurately determine the pose of the scanning device and, subsequently, losing the ability to generate data about the object subject of the scan (e.g., the surface or canal of the ear 306). The guidance system can provide the operator with guidance on: (a) maintaining the field of fiducial vision; and (b) conducting scanning sweeps of the surface to generate optimal data for reconstruction.

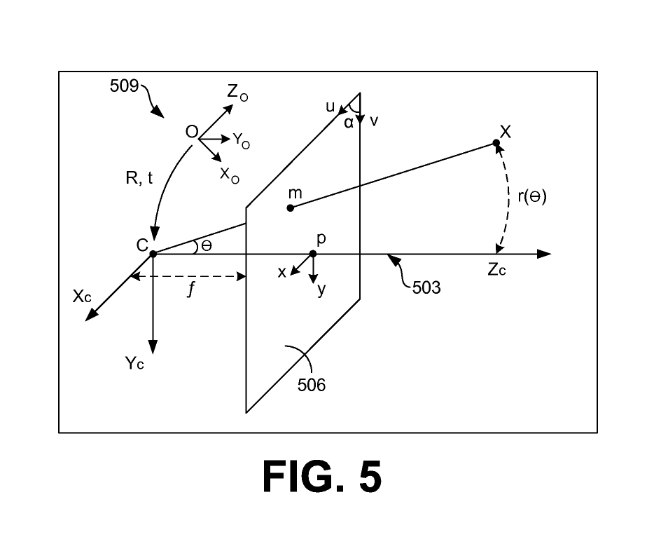

Referring next to FIG. 5, shown is a camera model that may be employed in the determination of world points and image points using one or more digital images captured via the imaging devices 115. By employing the camera model of FIG. 5, a mapping between rays and image points may be determined permitting the imaging devices 115 to behave as a position sensor. In order to generate adequate three-dimensional reconstructions of a surface cavity subject to a scan, a pose of a scanning device 100 relative to six degrees of freedom (6DoF) is beneficial.

Initially, a scanning device 100 may be calibrated using the imaging devices 115 to capture calibration images of a calibration object whose geometric properties are known. By employing the camera model of FIG. 5 to the observations identified in the calibration images, internal and external parameters of the imaging devices 115 may be determined. For example, external parameters describe the orientation and position of an imaging device 115 relative to a coordinate frame of an object. Internal parameters describe a projection from a coordinate frame of an imaging device 115 onto image coordinates. Having a fixed position of the imaging devices 115 on the scanning device 100, as depicted in FIGS. 1A-1C, permits the determination of the external parameters of the scanning device 100 as well. The external parameters of the scanning device 100 may be employed in the generation of three-dimensional reconstructions of a surface cavity subject to a scan.

In the camera model of FIG. 5, projection rays meet at a camera center defined as C, wherein a coordinate system of the camera may be defined as X_{c}, Y_{c}, Z_{c}, where Z_{c} is defined as the principal axis 503. A focal length f defines a distance from the camera center to an image plane 506 of an image captured via an imaging device 115. Using a calibrated camera model, perspective projections may be represented via:

$$( \begin{matrix}x \\ y \\ 1\end{matrix} ) ≃ [ \begin{matrix}f & 0 & 0 & 0 \\ 0 & f & 0 & 0 \\ 0 & 0 & 1 & 0\end{matrix} ] ( \begin{matrix}{X}_{c} \\ {Y}_{c} \\ {Z}_{c} \\ 1\end{matrix} ) . \qquad ( EQ . 1 )$$

A world coordinate system 509 with principal point O may be defined separately from the camera coordinate system, as X_{O}, Y_{O}, Z_{O}. According to various embodiments, the world coordinate system 509 may be defined at a base location of the probe 109 of the scanning device 100, however, it is understood that various locations of the scanning device 100 may be used as the base of the world coordinate system 509. Motion between the camera coordinate system and the world coordinate system 509 is defined by a rotation R, a translation t, and a tilt φ. A principal point p is defined as the origin of a normalized image coordinate system (x, y) and a pixel image coordinate system is defined as (u, v), wherein α is

$$( \frac{π}{2} )$$

in a conventional orthogonal pixel coordinate axes. The mapping of a three-dimensional point X to the digital image m is represented via:

$$\begin{matrix}m ≃ [ \begin{matrix}{m}_{u} & - {m}_{u} cot ( α ) & {u}_{0} \\ 0 & \frac{{m}_{v}}{sin ( α )} & {v}_{0} \\ 0 & 0 & 1\end{matrix} ] [ \begin{matrix}f & 0 & 0 & 0 \\ 0 & f & 0 & 0 \\ 0 & 0 & 1 & 0\end{matrix} ] [ \begin{matrix}R & t \\ 0 & 1\end{matrix} ] X \\ = [ \begin{matrix}{m}_{u} f & - {m}_{u} f cot ( α ) & {u}_{0} \\ 0 & \frac{{m}_{v}}{sin ( α )} f & {v}_{0} \\ 0 & 0 & 1\end{matrix} ] [ \begin{matrix}R & t\end{matrix} ] X\end{matrix} \qquad ( EQ . 2 )$$

Further, the camera model of FIG. 5 may account for distortion deviating from a rectilinear projection. Radial distortion generated by various lenses of an imaging device 115 may be incorporated into the camera model of FIG. 5 by considering projections in a generic model represented by:

r(θ)=1+k₂θ³+k₃θ⁵+k₄θ⁷+ (EQ. 3)

As EQ. 3 shows a polynomial with four terms up to the seventh power of θ, the polynomial of EQ. 3 provides enough degrees of freedom (e.g., six degrees of freedom) for a relatively accurate representation of various projection curves that may be produced by a lens of an imaging device 115. However, other polynomial equations with lower or higher orders or other combinations of orders may be used.

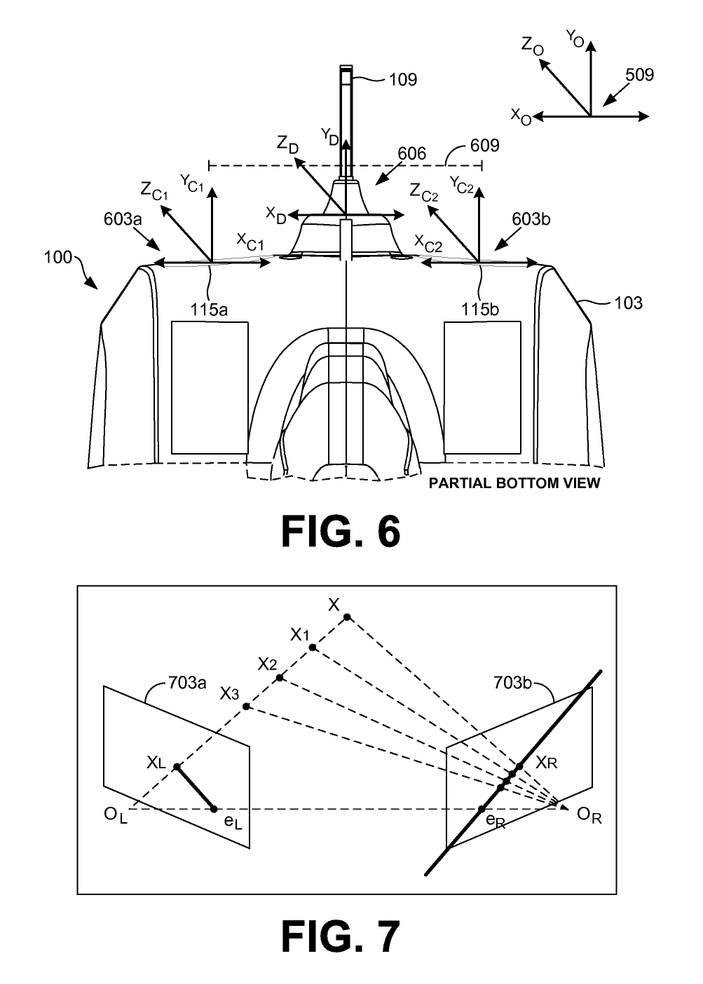

Turning now to FIG. 6, shown is another drawing of a portion of the scanning device 100 according to various embodiments. In the non-limiting example of FIG. 6, the scanning device 100 comprises a first imaging device 115a and a second imaging device 115b, all implemented in a fashion similar to that of the scanning device described above with reference to FIGS. 1A-1C. The first imaging device 115a and the second imaging device 115b may be mounted within the body 103 without hindering or impeding a view of the first imaging device 115a and/or the second imaging device 115b.

The placement of two imaging devices 115 permits computations of positions using epipolar geometry. For example, when the first imaging device 115a and the second imaging device 115b view a three-dimensional scene from their respective positions, geometric relations exist between the three-dimensional points and their projections on two-dimensional images that lead to constraints between the image points. These geometric relations may be modeled via the camera model of FIG. 5 and may incorporate the world coordinate system 509 and one or more camera coordinate systems, such as camera coordinate system 603a and camera coordinate system 603b (collectively camera coordinate systems 603).

By determining the internal parameters and external parameters for each imaging device 115 via the camera model of FIG. 5, the camera coordinate systems 603 for the imaging devices 115 may be determined relative to the world coordinate system 509. The geometric relations between the imaging devices 115 and the scanning device 100 may be modeled using tensor transformation (e.g., covariant transformation) that may be employed to relate one coordinate system to another. Accordingly, a device coordinate system 606 may be determined relative to the world coordinate system 509 using at least the camera coordinate systems 603. As may be appreciated, the device coordinate system 606 relative to the world coordinate system 509 comprises the pose estimate of the scanning device 100.

In addition, the placement of the two imaging device 115 in the scanning device 100 may be beneficial in implementing computer stereo vision. For example, both imaging devices 115 can capture digital images of the same scene; however, they are separated by a distance 609. A processor in data communication with the imaging devices 115 may compare the images by shifting the two images together over the top of each other to find the portions that match to generate a disparity used to calculate a distance between the scanning device 100 and the object of the picture. However, implementing the camera model of FIG. 5 is not as limited as an overlap between two digital images taken by a respective imaging device 115 and may not be warranted when determining independent camera models for each imaging device 115.

Moving on to FIG. 7, shown is the relationship between a first image 703a captured, for example, by the first imaging device 115a and a second image 703b, for example, captured by the second imaging device 115b. As may be appreciated, each imaging device 115 is configured to capture a two-dimensional image of a three-dimensional world. The conversion of the three-dimensional world to a two-dimensional representation is known as perspective projection, which may be modeled as described above with respect the camera model of FIG. 5. The point X_{L} and the point X_{R} are shown as projections of point X onto the image planes. Epipole e_{L} and epipole e_{R} have centers of projection O_{L} and O_{R} on a single three-dimensional line. Using projective reconstruction, the constraints shown in FIG. 7 may be computed.

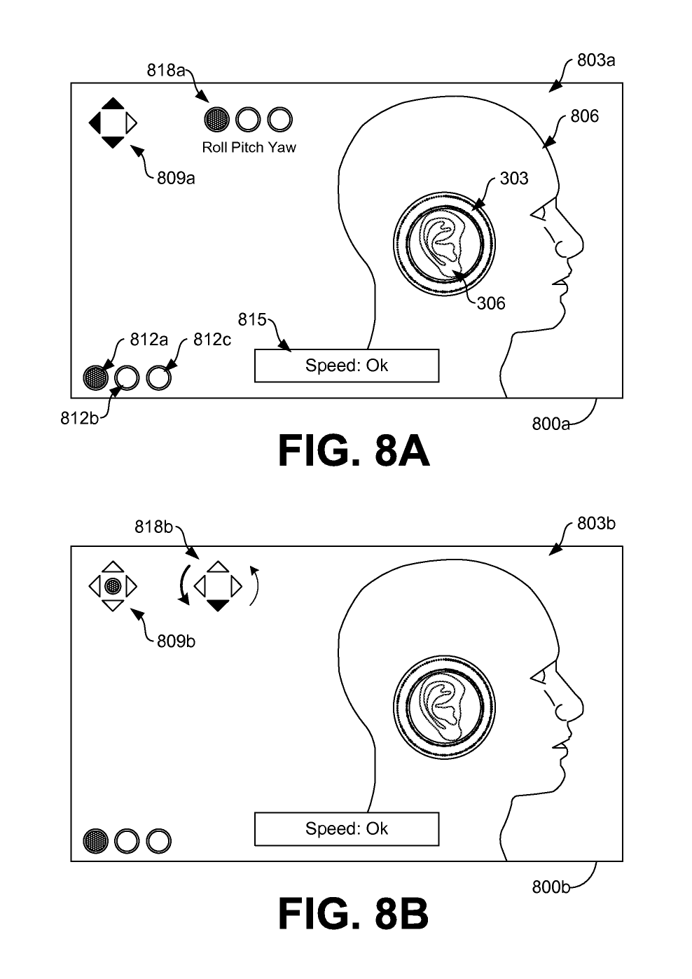

Referring next to FIG. 8A, shown is an example guidance user interface 800a that may be rendered on a display, such as the display screen 118 (FIG. 1A) mounted within the scanning device 100 (FIG. 1A). According to various embodiments, the guidance user interface 800a may be rendered on a display internal to the scanning device 100 or a display external to the scanning device 100, such as a television, a mobile device, or a computer monitor, independently or simultaneously, with a three-dimensional reconstruction as described in U.S. patent application Ser. No. 14/049,666 entitled “DISPLAY FOR THREE-DIMENSIONAL IMAGING,” which is hereby incorporated by reference in its entirety.

In the non-limiting example of FIG. 8A, a video feed 803a is generated for display in the guidance user interface 800a during a scan of an object, such as the ear 306 of a human being 806. As discussed above, the fiducial marker 303 may be positioned near or around the ear 306 to facilitate the determination of a pose of the scanning device 100, used in generating data about the surface or cavity being scanned by the scanning device 100. To this end, the pose of the scanning device 100 may be used in collecting data about the object subject to the scan as well as determining a change in the current motion to recommend to the operator to assist with the collection of the data.

As previously described, the guidance system of the scanning device 100 provides the operator with direction (or indications) on how to maintain the field of fiducial vision between the imaging devices 115 and the fiducial marker 303 as well as how to conduct scanning sweeps of the object in order to generate data about the object being subjected to the scan. Motion guidance can be provided to ensure that a complete set of data is collected, while avoiding adverse contact with the object being scanned. Accordingly, the guidance user interface 800 may employ a directional component 809 that visually depicts a directed movement of the scanning device 100. In the non-limiting example of FIG. 8A, the directional component 809 includes an up arrow, a down arrow, a left arrow, and a right arrow. The illumination of an arrow (e.g., the right arrow) instructs the operator of the scanning device to move the scanning device in that direction (e.g., to the right).

Independently, or in addition to, the directional component 809, the guidance user interface 800 may comprise a plurality of indicators 812a-c (collectively indicators 812) that may be used to assist the operator in maintaining a speed and/or a position of the scanning device 100. For example, when a respective one of the indicators 812 is emphasized or illuminated, the operator of the device may determine whether to maintain the speed of movement of the scanning device 100 and/or the position of the scanning device 100. As a non-limiting example, an indicator 812 may be assigned a color that, when illuminated or otherwise displayed, provides the operator with an indication of the performance of the scan.

As another non-limiting example, the indicator 812a may be assigned a red color, the indicator 812b may be assigned a yellow color, and the indicator 812c may be assigned a green color. When the indicator 812c is illuminated (e.g., green), the operator may be directed that the position (or speed or movement) of the scanning device 100 is accurate or ideal. When the indicator 812b is illuminated (e.g., yellow), it may indicate to the operator that the field of fiducial vision 403 (FIG. 4) may be lost, to proceed with caution, to slow the speed of movement of the scanning device 100, and/or to consult the directional component 809 on how to correct the position of the scanning device 100. When the indicator 812a is illuminated (e.g., red), it may advise the operator that the field of fiducial vision 403 has been lost, to stop the scan of the scanning device 100, to restart the scan using the scanning device 100, and/or to consult the directional component 809 on how to correct the position, speed, or movement of the scanning device 100. In various embodiments, the guidance user interface 800 may further comprise a fourth indicator 812 to direct the operator of the scanning device 100 to increase the speed or adjust the movement during the scan.

Independently, or in addition to, the above described components of the guidance user interface 800, the guidance user interface 800 may comprise a speed component 815 that provides guidance with respect to the speed of the scanning device 100 such that data may be optimally obtained. As a non-limiting example, during a scan of the ear canal, various sensors on the scanning device may be configured to obtain data about the ear canal, such as the shape and/or size of the ear canal. If the operator of the scanning device 100 were to pull the device out of the ear canal too fast, the sensors may not be able to collect an ideal amount of data to be used in generating a three-dimensional reconstruction of the ear canal. In addition, if the scanning device 100 were to scan the ear canal too slowly, the probability of obtaining redundant data is increased. Accordingly, the data and/or the amount of data collected by the scanning device 100 may be used in determining an optimal speed of the scanning device 100 and the speed component 815 may be updated according to the actual speed relative to the optimal speed. For example, speed feedback such as, but not limited to, “Ok,” “Good,” “Increase Speed,” “Decrease Speed,” “Stop,” “Start,” etc., may be indicated via the speed component 815. In addition, predetermined optimal speeds may be stored in logic or memory and used in generating the speed component 815 in the guidance user interface 800.

According to various embodiments, the speed of the scanning device 100 may be determined by periodically measuring the position of the scanning device 100 relative to an amount of time that has elapsed between the measurement of the position. In alternative embodiments, the data obtained during the scan may be used in determining the speed of the scanning device 100. For example, if the data being obtained by the scanning device 100 is indicative that the scanning device 100 is located in a particular region of the ear canal, the time taken for the scanning device 100 to move to a subsequent region may be used in determining the speed of the scanning device 100.

According to various embodiments, the guidance user interface 800 may further comprise a pitch-roll-yaw guidance component 818 that provides guidance of the scanning device 100 with respect to an additional three degree of freedom such that data may be optimally obtained for the item subject to the scan and guidance may be provided that lessens or avoids a probability of colliding with the object and/or losing the field of fiducial vision 403. For example, the pitch-roll-yaw guidance component 818 may be rendered in the guidance user interface 800 to provide the user with a recommended pitch, roll, and/or yaw of the scanning device 100 that, if followed, will lessen or avoid the probability of colliding with the object during the scan and/or losing the field of fiducial vision 403. To this end, the pitch-roll-yaw guidance component 818 may comprise three indicators, each of which can correspond to roll, pitch, and/or yaw, respectively. Each of the three indicators may notify the operator of the scanning device 100 whether the scanning device 100 is within an operational threshold of distance from the object subject to the scan.

For example, an indicator corresponding to a pitch may be assigned a green color if operating within the operational threshold, a yellow color if between the operational threshold and a collision or a loss of field of fiducial vision 403, and a red color if the pitch has caused the scanning device 100 to collide with the object or has caused a loss of the field of fiducial vision 403. When the indicator is illuminated (e.g., red), it may advise the operator that the field of fiducial vision 403 has been lost, to stop the scan of the scanning device 100, to restart the scan using the scanning device 100, and/or to correct the pitch of the scanning device 100. Similarly, the colors may be employed for a corresponding one of the indicators if a roll or yaw may cause a collision or a loss of the field of fiducial vision 403.

Moving on to FIG. 8B, alternative examples of the directional component 809b and the pitch roll yaw component 818b are shown. Similar to the directional component 809a of FIG. 8A, the directional component 809b can include an up arrow, a down arrow, a left arrow, and a right arrow. The illumination of an arrow (e.g., the right arrow) instructs the operator of the scanning device to move the scanning device in that direction (e.g., to the right). In addition to the arrows, a circular icon may represent whether to adjust a depth of the scanning device 100. For example, when performing a scan of an ear canal, the operator must avoid going too deep in the ear canal. Accordingly, the circular icon (or a similar icon) may be illuminated or otherwise emphasized to notify the operator to adjust the depth of the scanning device 100 and/or the probe 109 (FIG. 1) of the scanning device 100.

The pitch-roll-yaw guidance component 818 can provide guidance of the scanning device 100 with respect to an additional three degree of freedom such that data may be optimally obtained for the item subject to the scan and guidance may be provided that lessens or avoids a probability of colliding with the object and/or losing the field of fiducial vision 403. In the non-limiting example of FIG. 8B, the pitch-roll-yaw guidance component 818 may be rendered in the guidance user interface 800 to provide the user with a recommended pitch, roll, and/or yaw of the scanning device 100 that, if followed, will lessen or avoid the probability of colliding with the object during the scan and/or losing the field of fiducial vision 403. The pitch-roll-yaw guidance component 818 may comprise an up arrow and a down arrow (pitch up or down), a left arrow and a right arrow (yaw left or right), and a curved arrow (rotate left or right). Each of the arrows may notify the operator of the scanning device 100 whether the scanning device 100 is within an operational threshold of distance from the object subject to the scan.

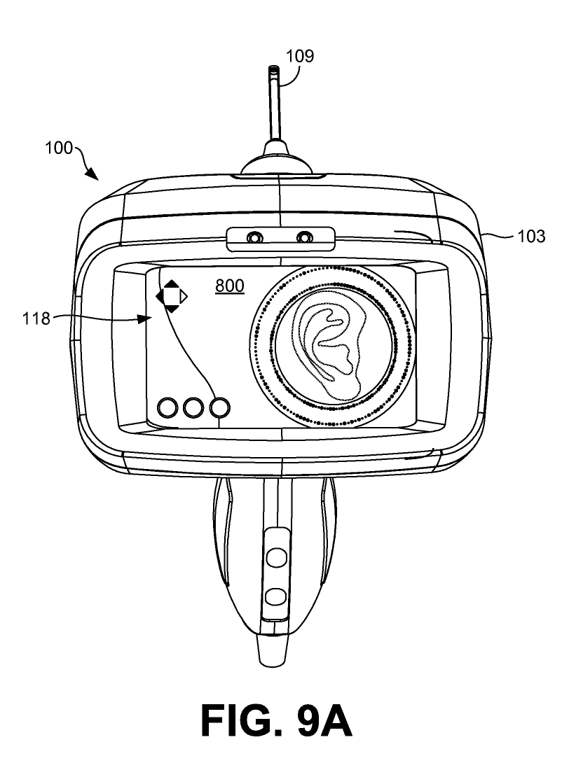

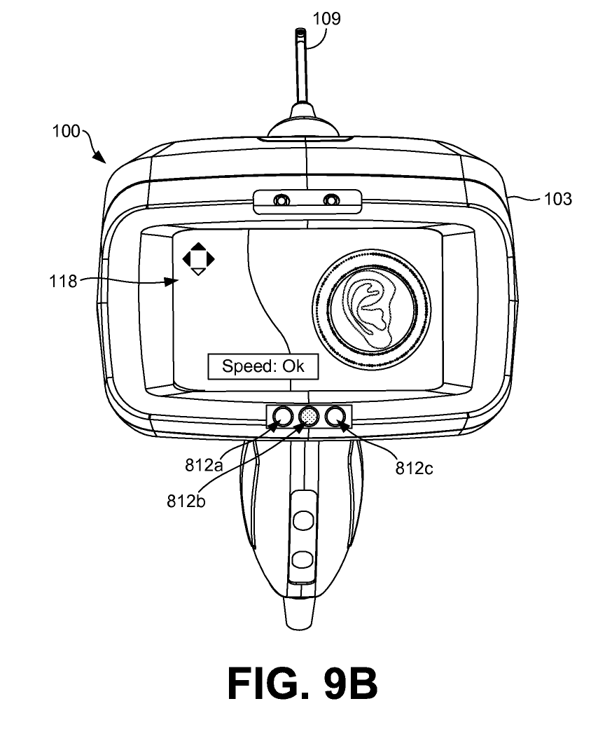

Referring next to FIG. 9A, shown is non-limiting example of a scanning device 100 with the guidance user interface 800 of FIGS. 8A-B rendered on the display screen 118 that is affixed to and/or mounted within the scanning device 100. In the non-limiting example of FIG. 9A, the guidance user interface 800 is rendered on the display screen 118 that is mounted on the opposite side of the body 103 from the probe 109 and distally from the hand grip 106. To this end, when an operator takes the hand grip 106 in the operator's hand and positions the probe 109 to scan a surface, both the probe 109 and the display screen 118 are easily visible to the operator. According to various embodiments, the display screen 118 may display, either separately or simultaneously, real-time constructions of three-dimensional images corresponding to the scanned cavity in association with the guidance user interface 800, for example, as described in co-pending U.S. patent application Ser. No. 14/049,666, entitled “DISPLAY FOR THREE-DIMENSIONAL IMAGING,” filed on Oct. 9, 2013, which is hereby incorporated by reference in its entirety.

Moving on to FIG. 9B, shown is another non-limiting example of a scanning device 100 rendering the guidance user interface 800 in the display screen 118 affixed to and/or mounted within the scanning device 100. However, in the non-limiting example of FIG. 9B, the indicators 812a-c are embodied in hardware on the scanning device as opposed to being graphically represented in the guidance user interface 800. To this end, the indicators 812a-c may comprise light-emitting-diodes (LEDs) or similar components that are configured to illuminate various colors that may be indicative of how to operate the scanning device 100.

As a non-limiting example, the indicator 812a may comprise a red LED, the indicator 812b may comprise a yellow LED, and the indicator 812c may comprise a green LED. As described above, when the indicator 812c is illuminated (e.g., green), the operator may be advised that the position of the scanning device 100 is accurate or ideal. Similarly, when the indicator 812b is illuminated (e.g., yellow), it may advise the operator that the field of fiducial vision 403 may be lost, to proceed with caution, to slow the speed of movement of the scanning device 100, and/or to consult the directional component 809 on how to correct the position of the scanning device. When the indicator 812a is illuminated (e.g., red), it may advise the operator that the field of fiducial vision 403 (FIG. 4) has been lost, to stop the scan of the scanning device 100, to restart the scan, and/or to consult the directional component 809 on how to correct the position of the scanning device 100. The illumination of the various indicators 812 may be controlled by one or more signals generated by, for example, a processor within the scanning device 100 executing a guidance system application, as will be described in greater detail below. Accordingly, when an operator takes the hand grip 106 in the operator's hand and positions the probe 109 to scan a surface, the probe 109, the display screen 118, and the indicators 812 are easily visible to the operator.

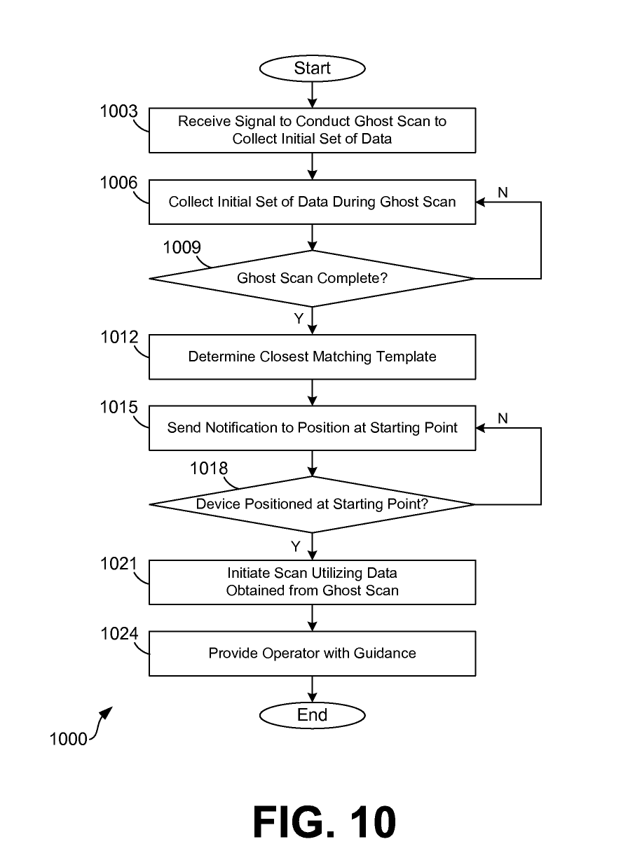

Referring next to FIG. 10, shown is a flowchart that provides one example of the operation of a portion of a guidance system application 1000 that may be executed by a processor, circuitry, logic, software executable in a processor, or any combination thereof, according to various embodiments. It is understood that the flowchart of FIG. 10 provides merely an example of the many different types of functional arrangements that may be employed to implement the operation of the portion of the guidance system application 1000 as described herein. As an alternative, the flowchart of FIG. 10 may be viewed as depicting an example of elements of a method implemented by a processor in data communication with a scanning device 100 (FIGS. 1A-1C) according to one or more embodiments.

Beginning with 1003, a signal is received by processing circuitry within the scanning device 100 to conduct an initial scan (also referred to as a “ghost scan”) during which an initial set of data is collected by the scanning device 100 to be employed by the guidance system application 1000 in guidance of the scanning device 100 as will be described herein. The signal received may be initiated by an operator of the scanning device 100 to provide notice that a scan of a surface is imminent so that the scanning device 100 may be operated to obtain a new set of data for a surface cavity. As a non-limiting example, an operator may engage a button, labeled e.g., “begin scan,” rendered on the display screen 118, or any other similar component, that may initiate a transmission of a signal from the display screen 118 to the processing circuitry. In alternative embodiments, the trigger 121 (FIG. 1A) of the scanning device 100 may be utilized by the operator to initiate the scan. As a non-limiting example, the operator may quickly depress and release the trigger 121 to initiate the scan.

In 1006, the guidance system application 1000 may collect an initial set of data during the ghost scan. As a non-limiting example, a ghost scan may comprise positioning the probe 109 (FIG. 1A) within the ear canal and slowly pulling the probe out of the ear canal. As may be appreciated, the initial set of data obtained during the ghost scan from multiple sensors within the scanning device 100 may comprise information about the object being subjected to the scan such as the size, shape, and/or other characteristics of the surface and/or cavity. According to various embodiments, the ghost scan may collect less data than a subsequent and/or more thorough scan of the object.

In 1009, it may be determined whether the ghost scan is complete. As a non-limiting example, it may be determined whether enough data has been obtained during the initial ghost scan to enable the guidance system, implemented in the scanning device 100, to accurately guide the operator of the scanning device 100. To this end, the initial set of data obtained during the ghost scan may be utilized by the guidance system application 1000 in calibrating and/or initiating the guidance system. If the ghost scan has not been completed (e.g., enough data has not been obtained during the ghost scan), the guidance system application 1000 may continue to collect data. As a non-limiting example, the scanning device, via the guidance system user interface 800, may prompt the operator to conduct an additional and/or replacement sweep of the object being subjected to the scan, such as the ear canal, as shown in 1006. To this end, a notification may be rendered in a guidance user interface 800 on the display screen 118 of the scanning device 100.

In 1012, if the ghost scan has been completed, a closest matching template of the object may be determined to be used in guidance of the scanning device 100 in a subsequent scan. To this end, a closest matching template may be determined by comparing data from the initial set of ghost scan data to predefined templates stored in memory. According to one embodiment, templates of ear canals may be stored in memory and may be utilized in guidance for scanning a particular ear canal. A “best match” template may be determined from the ghost scan and used in providing the operator with guidance. In various embodiments, the “best match” template may be modified to conform to the data generated during the ghost scan.

In 1015, the scanning device 100, via the guidance system user interface 800, may prompt or otherwise provide the user with a notification to position the scanning device 100 at a starting point or location. According to various embodiments, the directional component 809 (FIGS. 8A-B), the indicators 812 (FIG. 8A), and/or the pitch roll yaw component 818 (FIGS. 8A-B) may be utilized in providing the operator with direction as to the starting point for a scan. In 1018, it may be determined whether the scanning device 100 is positioned at the starting point to conduct a subsequent and more thorough scan of the object. According to various embodiments, the determination is made in 1018 by determining whether the fiducial marker is located within a field of fiducial vision with the imaging devices 115 (FIG. 1A) and, if so, determining the location of the scanning device 100 utilizing the fiducial marker as described in co-pending U.S. patent application Ser. No. 14/049,687, entitled “INTEGRATED TRACKING WITH WORLD MODELING,” filed on Oct. 9, 2013, and co-pending U.S. patent application Ser. No. 14/049,678, entitled “INTEGRATED TRACKING WITH FIDUCIAL-BASED MODELING,” filed on Oct. 9, 2013, both of which are hereby incorporated by reference in their entirety.

If the scanning device 100 is not positioned at the starting point, in 1015, then an additional notification may be generated to prompt the operator to position the device at the starting point at 1015. In an alternative embodiment, the notification sent in 1015 may remain, for example, in the guidance user interface 800 until the device is positioned at the starting point. As previously discussed, the directional component 809, the indicators 812, and/or the pitch roll yaw component 818 may be utilized in providing the operator with direction to the starting point for a scan. If the determination is made that the scanning device 100 is positioned at the starting point, a scan may be initiated utilizing the initial set of data obtained from the ghost scan, in 1021. In 1024, the operator of the scanning device 100 is provided with guidance via the guidance user interface 800, as discussed above with respect to FIGS. 8A-B and 9A-B.

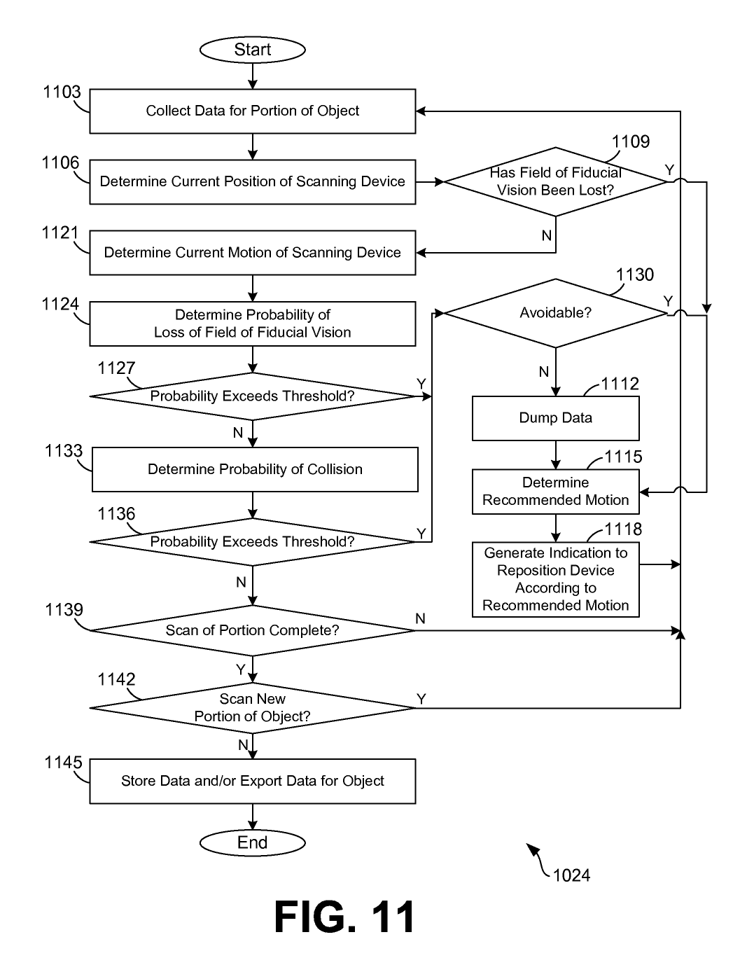

Moving on to FIG. 11, shown is a flowchart that provides one example of the operation of a portion of a guidance system application 1000 that may be executed by a processor, circuitry, logic, software executable in a processor, or any combination thereof, according to various embodiments. It is understood that the flowchart of FIG. 11 provides merely an example of the many different types of functional arrangements that may be employed to implement the operation of the portion of the guidance system application 1000 as described herein. As an alternative, the flowchart of FIG. 11 may be viewed as depicting an example of elements of a method implemented by a processor in data communication with a scanning device 100 (FIGS. 1A-1C) according to one or more embodiments.

Specifically, in FIG. 11, more detail is provided with respect to 1024 in which guidance is provided to an operator of the scanning device 100 after a scan of an object is initiated in 1021 (FIG. 10). As a non-limiting example, an ear canal or any other surface or cavity may comprise multiple sections. For example, the ear canal may comprise the tympanic membrane, the auditory external canal, etc. Utilizing data collected by the sensors of the scanning device 100, a particular section of the ear canal being scanned may be determined and guidance provided to the operator may be specific to the particular section. For example, the guidance system application 1000 may determine and indicate that the operator is to conduct a relatively quick sweep of the tympanic membrane and a relatively slow sweep of the auditory external canal. This provides the benefit of optimizing data generation in specific areas of the object being subjected to the scan.

Beginning with 1103, data is collected and/or stored in association with coordinates identifying a respective portion of the object being subjected to the scan. For example, the probe 109 (FIG. 1) may be configured to emit a circular light to collect data about the size, shape, or structure of a region of an ear canal based on the reflection of the fan light produced by the probe 109. As the respective portion of the ear canal is being scanned, data associated with the respective portion may be collected and/or stored in association with coordinates identifying the respective portion. For example, data collected in a scan of the auditory external canal may be stored in association with auditory external canal data that may be used in generating an accurate three-dimensional reconstruction of the auditory external canal. According to various embodiments, storage of the data may comprise placement of the data in memory internal to the scanning device 100 or external to the scanning device 100, such as flash or network storage.

Next, in 1106, the position of the scanning device 100 is determined relative to the object being subjected to the scan in a three-dimensional space. As described above, the position of the scanning device 100 may be determined utilizing one or more sensors in communication with the scanning device 100 as described in co-pending U.S. patent application Ser. No. 14/049,687, entitled “INTEGRATED TRACKING WITH WORLD MODELING,” filed on Oct. 9, 2013, and co-pending U.S. patent application Ser. No. 14/049,678, entitled “INTEGRATED TRACKING WITH FIDUCIAL-BASED MODELING,” filed on Oct. 9, 2013, both of which are hereby incorporated by reference in their entirety.

As may be appreciated, if the scanning device 100 loses the field of fiducial vision 403 with the fiducial marker 403, the position of the tracking device 100 is unable to be determined. Thus, in 1109, it is determined whether the field of fiducial vision has been lost. If the field of fiducial vision has been lost, the process may proceed to dump that data previously collected (1112), determine a recommended motion (e.g., change in the current motion) for the scanning device 100 (1115), and generate an indication to reposition the scanning device (1118) in view of the recommended motion. The process then proceeds to 1103 to continue collection of data for the portion of the object being subjected to the scan.

If the field of fiducial vision has not been lost, a current position of the scanning device has been determined in 1106. In 1121, a current motion may be determined utilizing at least the current position of the scanning device 100 and a past motion of the scanning device 100. According to various embodiments, the current motion of the scanning device 100 can be determined a projected movement of the scanning device 100 along a projected course that is determined using the past motion and/or the pose of the scanning device 100. In various embodiments, the past motion is determined by periodically measuring positions or points of the scanning device 100 in a three-dimensional space relative to an amount of time that has elapsed between the measurements of the positions. These positions or points can be used to determine a projected course that the scanning device 100 likely will follow if the scanning device 100 continues its motion beyond the past motion. In alternative embodiments, the data obtained during the scan may be used in determining the speed of the scanning device 100. For example, if the data being obtained by the scanning device 100 is indicative that the scanning device 100 is located in a particular region of the ear canal, the time taken for the scanning device 100 to move to a subsequent region may be used in determining the past motion of the scanning device 100 as well as the projected motion.

As may be appreciated, an operator of the scanning device 100 may accidently and erroneously collide with a fiducial marker 303, such as the one shown circumnavigating the surface of the ear 306 in FIG. 3. Similarly, the operator of the scanning device 100 may accidently position the scanning device 100 such that the imaging devices 115 (FIG. 1A) lose the field of fiducial vision 403 (FIG. 4) with the fiducial marker 303. Accordingly, a “violation of fiducial space” may comprise a collision, a high probability of a collision, a loss of the field of fiducial vision 403, or a high probability of a loss of the field of fiducial vision 403 during a scan. The current motion may be indicative of whether the operator of the scanning device 100 may result in a violation of the fiducial space between the scanning device 100 and the fiducial marker 303. Accordingly, in 1124, a probability that the current motion will cause of a loss of the field of fiducial vision 403 can be determined, for example, by comparing the current motion to the location of the fiducial marker 303 and/or the human being 806 (FIG. 8A).

In 1127, if it is determined that the probability meets and/or exceeds a predefined threshold, then the current motion will likely cause a loss of field of fiducial vision 403. It can then be determined, at 1130, whether a loss of the field of fiducial vision is avoidable. If the loss of the field of fiducial vision 403 is unavoidable the process may proceed to dump that data previously collected (1112), determine a recommended motion (e.g., a change in the current motion) for the scanning device 100 (1115), and generate an indication to reposition the scanning device (1118) according to the recommended motion. If the loss of the field of fiducial vision 403 is avoidable, the process may proceed to determine a recommended motion for the scanning device 100 (1115) that may avoid the loss of the field of fiducial vision 403, as well as generate an indication to reposition the scanning device (1118) or change the current motion of the scanning device 100 according to the recommended motion.

As may be appreciated, the recommended motion determined in 1115 may be along a projected course that, if relatively followed by the operator, may avoid the loss of the field of fiducial vision 403. The recommended motion (e.g., the recommended change in the current motion) may be determined, for example, by comparing the last known location of the fiducial marker 303 to the current position of the scanning device 100. In 1118, an indication to reposition or change the current motion of the scanning device 100 may be generated to prompt the operator to adjust movement of the scanning device 100 according to the recommended motion. According to various embodiments, the recommended change in the current motion or the repositioning can be recommended to the operator as described above with respect to FIGS. 8, 9A, and 9B. For example the recommended motion may be suggested to the user utilizing at least the directional component 809 (FIG. 8A), the indicators 812 (FIGS. 8A-B), the speed component 815 (FIG. 8A), and/or the pitch roll yaw guidance component 818 (FIGS. 8A-B).

Referring back to 1127, if it is determined that the probability of a loss of field of fiducial vision 403 does not exceed the threshold, the process may proceed to 1133 where, a probability that the current motion will cause a collision between the scanning device 100 and another object (e.g., the fiducial marker 303 or the human being 806) may be determined. As a non-limiting example, two or more positions of the scanning device 100 along the past motion and the speed of the current motion of the scanning device 100 may be used to determine that an imminent collision can occur. In some cases, the probability may be relatively low. Thus, in 1136, it may be determined whether the probability exceeds a particular threshold, such as a predefined threshold stored in memory or logic.

If the probability meets and/or exceeds the particular threshold, the process may move to 1130 to determine whether the collision is avoidable. If the collision is avoidable, a recommended motion may be determined utilizing the current position of the scanning device 100, the current motion, etc., as described above in 1115. A recommended change in the current motion can be along a projected motion that, if followed by the operator, may avoid the collision. In 1118, an indication of the recommended motion can be presented to the operator. According to various embodiments, the recommended motion and/or the suggested repositioning of the scanning device 100 may be presented to the operator as described above with respect to FIGS. 8A-B and 9A-B. Referring back to 1130, if the collision is unavoidable, the process may proceed to dump that data previously collected (1112), determine a recommended motion for the scanning device 100 (1115), and generate an indication to change the current motion and/or reposition the scanning device (1118) according to the recommended motion.

If the probability that the current motion will cause a loss of the field of fiducial vision 403 does not meet and/or exceed the particular threshold, in 1139, it can be determined whether the scan of the respective portion of the object has been completed by the operator (e.g., enough data has been collected during one or more sweeps of the respective portion of the surface or cavity). If the scan of the portion has not been completed, the process may return to 1103 to continue the scan of that portion.

If the scan of the portion of the object is complete (1139), then it may be determined whether the scanning device 100 is conducting a scan of a new portion of the object in 1142. For example, the data being generated by the sensors of the scanning device 100 may indicate that a new portion of the object is being scanned. The data may indicate that, for example, the position of the probe 109 of the scanning device 100 has moved from the tympanic membrane to the auditory external canal. If the scanning device 100 is conducting a scan of a new portion of the object the process moves back to 1103 to continue collecting data for that respective portion of the object of the scan.

If the data collected is indicative that a new portion of the object is being scanned, in 1103, then data for the new portion of the object may be collected in association with the new portion. Accordingly, data may be obtained for respective portions of the object being scanned that may be beneficial, for example, in generating three-dimensional reconstructions specific to each portion or region of the object. Alternatively, if the scanning device 100 is not scanning a new portion of the object, then in 1145 the data collected for the one or more portions of the object can be stored in and/or exported from the scanning device 100. According to various embodiments, the data may comprise measurements taken for varying regions of an ear canal that may be beneficial in generating real-time three-dimensional reconstructions of the ear canal.

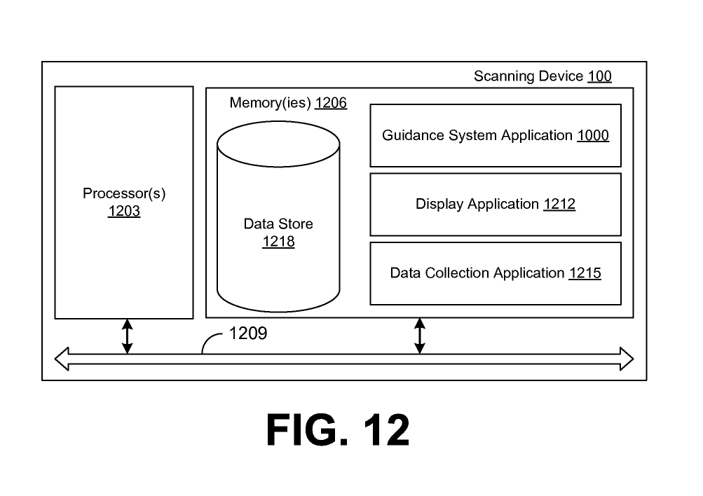

With reference to FIG. 12, shown is a schematic block diagram of a scanning device 100 according to an embodiment of the present disclosure. A scanning device 100 may comprise at least one processor circuit, for example, having a processor 1203 and a memory 1206, both of which are coupled to a local interface 1209. The local interface 1209 may comprise, for example, a data bus with an accompanying address/control bus or other bus structure as can be appreciated.

Stored in the memory 1206 are both data and several components that are executable by the processor 1203. In particular, the guidance system application 1000 may be stored in the memory 1006 and executable by the processor 1203, as well as other applications such as a display application 1212 configured to generate and render the guidance user interface 800 (FIGS. 8A-B) and a data collection application 1215 configured to collect and analyze data obtained from the various sensors of the scanning device 100. Also stored in the memory 1206 may be a data store 1218 and other data. In addition, an operating system may be stored in the memory 1206 and executable by the processor 1203.

It is understood that there may be other applications that are stored in the memory 1206 and are executable by the processor 1206 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 1006 and are executable by the processor 1203. In this respect, the term “executable” means a program file that is in a form that can ultimately be run by the processor 1203. Examples of executable programs may be, for example, a compiled program that can be translated into machine code in a format that can be loaded into a random access portion of the memory 1206 and run by the processor 1203, source code that may be expressed in proper format such as object code that is capable of being loaded into a random access portion of the memory 1206 and executed by the processor 1203, or source code that may be interpreted by another executable program to generate instructions in a random access portion of the memory 1206 to be executed by the processor 1203, etc. An executable program may be stored in any portion or component of the memory 1206 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1206 is defined herein as including both volatile and nonvolatile memory and data storage components. Volatile components are those that do not retain data values upon loss of power. Nonvolatile components are those that retain data upon a loss of power. Thus, the memory 1206 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1203 may represent multiple processors 1203 and/or multiple processor cores and the memory 1206 may represent multiple memories 1206 that operate in parallel processing circuits, respectively. In such a case, the local interface 1209 may be an appropriate network that facilitates communication between any two of the multiple processors 1203, between any processor 1203 and any of the memories 1206, or between any two of the memories 1206, etc. The local interface 1209 may comprise additional systems designed to coordinate this communication, including, for example, performing load balancing. The processor 1203 may be of electrical or of some other available construction.

Although the pose estimate application guidance system application 1000, the display application 1212, the data collection application 1215, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart of FIGS. 10 and 11 show the functionality and operation of an implementation of portions of the guidance system application 1000. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 1203 in a computer system or other system. The machine code may be converted from the source code, etc. If embodied in hardware, each block may represent a circuit or a number of interconnected circuits to implement the specified logical function(s).

Although the flowcharts of FIGS. 10 and 11 show a specific order of execution, it is understood that the order of execution may differ from that which is depicted. For example, the order of execution of two or more blocks may be scrambled relative to the order shown. Also, two or more blocks shown in succession in FIGS. 10 and 11 may be executed concurrently or with partial concurrence. Further, in some embodiments, one or more of the blocks shown in FIGS. 10 and 11 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the guidance system application 1000, the display application 1212, and the data collection application 1215, that comprises software or code can be embodied in any non-transitory computer-readable medium for use by or in connection with an instruction execution system such as, for example, a processor 1203 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the guidance system application 1000, the display application 1212, and the data collection application 1215, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same scanning device 100, or in multiple computing devices in a common computing environment. Additionally, it is understood that terms such as “application,” “service,” “system,” “engine,” “module,” and so on may be interchangeable and are not intended to be limiting.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A system, comprising:
a mobile scanning device configured to scan an object; and
a guidance system application executable by at least one processor, the guidance system application comprising logic that:
determines a position of the mobile scanning device in a three-dimensional space relative to the object of the scan utilizing at least a fiducial marker detected via at least one sensor of the mobile scanning device, wherein the fiducial marker is in a field of fiducial vision of the at least one sensor;
determines a current motion of the mobile scanning device during a scan of the object; and
generates an indication of a change in the current motion with respect to the object of the scan, the change in the current motion generated utilizing at least the position of the mobile scanning device in the three-dimensional space and the current motion of the mobile scanning device.

2. The system of claim 1, wherein the indication of the change in the current motion is generated in response to a probability that the at least one sensor will lose the field of fiducial vision based upon a predefined threshold.

3. The system of claim 2, wherein the guidance system application further comprises logic that determines the probability utilizing at least the current motion of the mobile scanning device.

4. The system of claim 1, wherein the indication of the change in the current motion is generated in response to a probability that the mobile scanning device will collide with the object subject to the scan based upon a predefined threshold.

5. The system of claim 2, wherein the change in the current motion reduces a probability of the at least one sensor losing the field of fiducial vision.

6. The system of claim 1, wherein the at least one sensor further comprises at least one imaging device.

7. The system of claim 1, wherein the mobile scanning device further comprises an otoscanner and the object being subjected to the scan further comprises a human ear canal.

8. A method, comprising:
tracking, by a computing device, a current position of a scanning device in a three-dimensional space relative to an object being subjected to a scan utilizing at least a fiducial marker detected via at least one sensor of the scanning device;
determining, by the computing device, a current motion of the scanning device during the scan of the object; and
generating, by the computing device, an indication of a change in the current motion utilizing at least the position of the scanning device in the three-dimensional space and the current motion of the scanning device.

9. The method of claim 8, wherein the indication of the change in the current motion is generated in response to a probability that the scanning device will collide with the fiducial marker based upon a predefined threshold.

10. The method of claim 9, further comprising determining, by the computing device, the probability utilizing at least the current motion of the scanning device.

11. The method of claim 8, wherein the indication of the change in the current motion is generated in response to a probability that the at least one sensor will lose a field of fiducial vision with the fiducial marker based upon a predefined threshold.

12. The method of claim 8, wherein the fiducial marker further comprises a circle-of-dots pattern.

13. The method of claim 8, wherein the change in the current motion is generated for a respective portion of the object being subjected to the scan.

14. The method of claim 8, wherein the at least one sensor further comprises at least one imaging device.

15. The method of claim 8, wherein the scanning device further comprises an otoscanner and the object being subjected to the scan further comprises a human ear canal.

16. A non-transitory computer-readable medium embodying a program executable in a processor in data communication with an otoscanner, the program comprising code that, when executed, causes the processor to:
determine a plurality of positions of the otoscanner in a three-dimensional space relative to the object of a scan utilizing at least a fiducial marker detected via at least one sensor of the otoscanner, wherein the fiducial marker is in a field of fiducial vision with the at least one sensor of the otoscanner;
determine a current motion of the otoscanner utilizing the plurality of positions of the otoscanner and a speed of motion of the otoscanner; and
generate an indication of a change in the current motion for the object subject the scan, the change in the current motion generated utilizing at least the plurality of positions of the otoscanner in the three-dimensional space, the current motion of the mobile scanning device, and the speed of motion of the otoscanner, wherein the indication is configured to be shown in association with the otoscanner during the scan.

17. The non-transitory computer-readable medium of claim 16, wherein the indication of the change in the current motion is generated in response to a probability that the at least one sensor will lose the field of fiducial vision based upon a predefined threshold.

18. The non-transitory computer-readable medium of claim 17, wherein the indication of the change in the current motion is generated in response to a probability that the at least one sensor will collide with the object subject to the scan or the fiducial marker based upon a predefined threshold.

19. The non-transitory computer-readable medium of claim 16, wherein the object being subjected to the scan further comprises a human ear canal.

20. The non-transitory computer-readable medium of claim 16, wherein the at least one sensor further comprises at least one imaging device.
